(19) United States
(12) Patent Application Publication
(10) Pub. No.: US 2009/0239935 A1
(43) Pub. Date: Sep. 24, 2009

(54) RNA-HELICASE AS A MARKER FOR RARE TUMORS

(75) Inventors: Kathrin Zeller, Bad Camenberg (DE); Sven Laarmann, Hamm (DE); Karin Mittmann, Munster (DE); Christoph Block, Datum (DE); Ralf Janknecht, Rochester, MN (US)

Correspondence Address: HENRY M FEIEREISEN, LLC;HENRY M FEIEREISEN, 708 THIRD AVENUE, SUITE 1501, NEW YORK, NY, 10017, US

(73) Assignees: Signalomics GMBH, Wien (AT); Mayo Foundation for Medical Education and Research, Rochester, MN (US)

(21) Appl. No.: 12/097,662

(22) PCT Filed: Dec. 14, 2006
(86) PCT No.: PCT/EP06/12042

§ 371 Date: Nov. 18, 2008

Related U.S. Application Data
(60) Provisional application No. 60/750,543, filed on Dec. 14, 2005.

(51) Int. Cl. A61K 31/7088; C12Q 1/26

(52) U.S. Cl. 514/44 A; 435/25

(57) ABSTRACT

Method for the diagnosis of diseases in a mammal, preferably in a human, wherein a probe from the mammal is examined with a view to an elevated level of RNA helicase and the elevated level of RNA helicase indicates the disease. The disease refers to esophagus carcinoma, pancreas carcinoma, stomach carcinoma, hepatocellular carcinoma, hepatoblastoma and cholangiocellular carcinoma.

### BACKGROUND INFORMATION

RNA-helicases form a large super family of conserved proteins, which take over many functions in biochemical processes, in which RNA-(ribonucleic acids) molecules take part, for example within the scope of transcription, splicing, transport, translation, RNA-digest and ribosome-biogenesis. The RNA-helicase for example, can effect the unraveling of duplex-RNA or can effect the disruption of the RNA-protein-interaction. In addition, the RNA-helicases can be involved in gene transcription. it was shown that they function as transcriptional cofactors [Abdelhaleem, 2005].

As the expression of key proteins, which contribute to the regulation of cell growth, proliferation, differentiation and cell death, takes place at the translation level and is thereby controlled partly by RNA-helicases [Abdelhaleem, 2004] one has to start from the position that there is also a connection between the translational regulation und the cancer genesis or its progression. For example, the RNA-helicase Rck/p54 is found to be overexpressed in colorectal-carcinomas and adenomas as well as in other tumors and tumor cell lines. Furthermore, the DDX1 RNA-helicase gene oftentimes is coamplified with N-myc in retinoblastoma and neuroplastoma, where a correlation of the coamplification is discussed in connection with a worse prognosis [Abdelhaleem, 2004].

P68 (DDX5) and p82 (DDX17) and its isoform p72 which is generated through alternative splicing are closely related members of the DEAD-box family of the RNA-helicases and are characterized by a conserved motif containing the sequence asp-glu-ala-asp (D-E-A-D). The proteins have detected ATPase, RNA-binding- and helicase activities. Besides its role in the RNA metabolism, for example, the p68 RNA-helicase is active in the regulation of gene transcription [Abdelhaleem, 2004]. It interacts with the estrogen-receptor-a (ER-a, estrogen receptor-a) and thus stimulates the ER-a dependent transcription. In addition, interactions take place between p68 and AIB1 (amplified in breast cancer), a steroid receptor-coactivator, which is predominantly overexpressed in breast tumors [Anzick et al. 19917] and with SRA (steroid receptor RNA-activator) a cofactor which functions as a RNA [Lanz et al., 1999].

As a starting point, the p68 RNA-helicase, SRA and AIB1 stimulate synergistically the ER-a dependent transcription [Watanabe et al. 2001]. In addition, p68 influences probably the transcription by interacting with the RNA-polymerase II and furthermore stimulates the transcription coactivators CBP and p300 [Rossow and Janknecht].

The homologous protein CBP and p300 are provided with an acetyl transferase activity and thus in the position to adjust the chromatin-structure and the function of a variety of different transcription factors. Rare diseases are diseases which are also named “orphan” or “rare diseases. Rare diseases are diseases which are afflicting fewer that 2000, which in Germany would amount to fewer than 40,000 patients and in the US would be fewer than 200,000 patients. In total. there are about 6000-7000 rare diseases of which about 1000 are known to be rare tumors or tumor sub-types.

Esophagus-carcinomas are malignant epithelial tumors of the esophagus that is, the upper gastro-intestinal tract, and transition to the stomach. These tumors are up to 96% squamous cell carcinomas and adenocarcinomas. Within the EU, the incidence of these is between 3-10/100,000, in Germany the esophagus carcinoma belongs to the rare carcinomas with increasing trend. The 5-year survival rate in the Unites States is 14%. The mortality data for Germany are 4/1000 and are approximately as high as the incidence, whereby annually 4000 patients die from esophagus carcinoma. While mortality rates have hardly changed, there is however a dramatic shift in the frequency of the two histological subtypes. In the 70's the portion of the adenocarcinoma were below 10%, but in the meantime, since 1990, that type of tumor has surpassed the numbers of the squamous carcinomas in the USA. Comparable developments are also observed in many centers in Europe and in Germany.

Normally, the diagnosis is carried out histologically by means of taking several esophagiascopy biopsies. Using a conventional histology with haematolixin/eosin-staining on paraffin slices, a differentiation between squamous carcinoma and adenocarcinoma, as well as also very rare small-celled carcinomas can be realized. Additional staining methods of the esophagus mucous membrane are helpful in the discovery of macroscopically non-distinct early carcinomas or multi-centric carcinomas. Special tumor markers or molecular-genetic markers for the esophagus carcinoma are so far not known.

A continuous increase in the incidence of pancreatic carcinoma, especially in the western hemisphere can be observed. Today, pancreas carcinoma represents the fifth most often occurring tumor-induced reason of death. Annually, there are 27,000 deaths in the USA and in Europe 50,000. Because of the difficult diagnosis, the aggressiveness of the course of the disease and the unsatisfying efficacy of systemic therapies, only 1-5% of all patients having adenocarcinoma of the endocrine pancreas are still alive 5 years after diagnosis. Accordingly, the incidence of the malignoma is approximately the mortality rate. This dismal 5-year survival rate is the worst of all tumor diseases in USA and Europe.

When pancreas carcinoma is suspected, a sonographic exam is carried out of the gall bladder and the pancreas. In order to eliminate other diseases, an x-ray contrast examination of the upper gastro-intestinal tract is carried out or even better, an esophagusgastroduodenoscopy. The magnetic resonance-cholangiopancreatography (MRCP) is one of the best non-invasive image-producing methods in order to illustrate stenoses of the pancreas or dilation of the gall duct. The best image-producing evaluation of the pancreas is effected through endosonography.

Until now the laboratory medical parameters have played only a subordinate role in diagnostics. Certain tumor markers such as CA 19-9, CA 125 and CEA together with the anamnesis and the clinical picture can serve to solidify the suspicion; however, they have no meaning in securing the diagnosis. Molecular genetic diagnostic methods, such as for example, the test of mutated k-ras in the pancreas secretion have a high sensitivity, however only an average specificity, such that it cannot be used for securing a routine diagnosis.

The frequency of the stomach carcinoma is decreasing worldwide. In 1995 20,000 patients suffered from a stomach carcinoma. The relative survival rate with 28% (in USA 22%) is in the lower third of the 5-year survival rates in tumor diseases. The localization of the tumors has changed likewise in the last few years; formerly they occurred predominantly in the distal area of the stomach, presently they are more in the proximal stomach and at the gastroesopageal transition.

At the start of the diagnosis an endoscopy with a biopsy is carried out, which detects 94% of the stomach-carcinomas. There are no specific serum-tumor markers for stomach carcinoma. Thus, CA72-4 and CEA can be utilized only at a progressed disease for use as a therapy control. Molecular genetic markers are not utilized in the diagnostics of stomach carcinomas.

The hepatocellular carcinoma (HCC) is a very rare childhood tumor. It appears in Western Europe and the USA with an incidence of 1-5/100,000 persons. Primary hepatic tumors are about 1-1.5% of all pediatric neoplasms and at an age up to 1.5 years has an incidence of 1.5/100,000 children. The hepatoblastoma is the most frequently occurring primary liver tumor in this age group followed by HCC. It occurs generally in adolescents. In the adult population HCC forms the most frequent liver carcinoma. Main symptoms is a palpable liver mass, stomach aches and in progressed stage, cachexia and icterus. The serum-alpha-fetoprotein is frequently elevated. In children the HCC can occur as a complication of a viral hepatitis especially in endemic areas but also as consequence of metabolic diseases. An HCC in an otherwise healthy liver is seen sooner in childhood age than in adults. The HCC is extraordinarily chemoresistant and when diagnosed is already in a progressive stage. Accordingly, the 5-year survival rate is only about 6%.

In the hepatocellular carcinoma, the tumor marker alpha1-fetoprotein is known to be a sensitive and specific serum marker for the presence of a hepatocellular carcinoma, whereby hepatitis-C diseases and chronic liver diseases are also influencing those markers. In combination with the detection of isoenzymes of gamma glutamyltranspeptidase or the abnormal plasma-prothrombin, the sensitivity can be raised to 78%.

The cholangiocellular carcinoma differs basically form hepatocelluar carcinoma as it is derived not from the hepatocytes but is derived form the gall duct epithelium. Pathogenically, is corresponds rather to an adenocarcinoma than a hepatocellular carcinoma, whereby in some instances mixed tumors are diagnosed. There is no connection with viral hepatides, liver cirrhosis or hepatic metabolic defects. Cholangiocellular carcinomas occur frequently in connection with chronic-inflammatory colon diseases and primary sklerosing cholangitis and have a bad prognosis.

The mortality for the hepatocelluar and the cholangiocellular carcinoma is very high, in the United States there is only a 6% 5-year survival rate. To date, for the cholangiocellular carcinoma no molecular genetic markers are utilized in the diagnosis.

It is thus an object of the present invention to provide a method for the diagnosis and treatment of tumors, especially the esophagus carcinoma, the pancreas carcinoma, the stomach carcinoma, the hepatocelluar carcinoma as well as the hepatoblastoma and the cholangiocelluar carcinoma in mammals, specifically in humans.

### SUMMARY

The present invention refers to methods and materials for the diagnosis and for the treatment of tumors, preferably esophageal carcinoma, pancreatic carcinoma, stomach carcinoma, hepatocellular carcinoma as well as hepatoblastoma and cholangiocellular carcinoma in mammals. The present invention also refers to a method for the production of corresponding diagnostics. By means of the present invention further molecules can be identified, which inhibit the activity of RNA-helicases (e.g. p68, p72 and/or p82 RNA-helicase). The invention also refers to a method for the treatment of cancer (e.g. esophageal carcinoma, pancreatic carcinoma, stomach carcinoma, hepatocellular carcinoma, hepatoblastoma, cholangiocellular carcinoma). The methods and materials that are provided herein are utilized for the diagnosis in mammals that have an esophageal carcinoma, pancreatic carcinoma, stomach carcinoma, hepatocellular carcinoma, hepatoblastoma, cholangiocellular carcinoma. The diagnosis of this form of cancer provides physicians with an earlier treatment tool for mammals than without the diagnosis.

Methods and materials, that are provided her can also be utilized for the identification of molecules, which inhibit a helicase (e.g. p68, p72 and/or p82 RNA-helicase). Such molecules can be used to treat mammals carrying cancer cells (e.g. esophageal carcinoma, pancreatic carcinoma, stomach carcinoma, hepatocellular carcinoma, hepatoblastoma, or a cholangiocellular carcinoma) such that the number of cancer cells can be reduced (e.g. 10, 20, 30, 40, 50, 60, 70, 8-, 90 or 100% reduction).

Generally, a method is emphasized in order to determine whether or not a mammal has cancer (e.g. esophagus carcinoma, pancreas carcinoma, stomach carcinoma, hepatocellular carcinoma, hepatoblastoma, cholangiocellular carcinoma but not mammary carcinoma). The method includes the detection whether a probe from a mammal shows an elevated level of a RNA-helicase (e.g. p68, p72 and/or p82 RNA-helicase) polypeptide, wherein the presence of an elevated level shows that the mammal has cancer (e.g. esophageal carcinoma, pancreatic carcinoma, stomach carcinoma, hepatocellular carcinoma, hepatoblastoma, cholangiocellular carcinoma). The mammal can be a human or can be another mammal, for example a horse or a pig. The probe can be a tissue probe form the organ to be diagnosed (suspected organ). The term “probe’ includes one or more tissue samples from different locations of the organ to be investigated or from the reference organ. In the case of a pancreatic carcinoma, a probe from the pancreas, in stomach carcinoma, a probe from the stomach, in hepatocelluar carcinoma, hepatoblastoma or cholangiocelluar carcinoma a probe each from the liver.

The method of the present invention for the diagnosis of tumors is based on a qualitative and/or quantitative test of one or more helicases in a tissue probe or in probes that have been derived therefrom (e.g. tissue lysates or similar). Preferably, they are helicases of the DEAD-box family that are known to the person skilled in the art. In an especially preferred embodiment an amount of at least one helicase from the group p68, p72 and/or p82 RNA-helicase are being tested. The diagnostic method according to the present invention accordingly is based on the use of RNA-helicases as tumor-associated markers.

The investigated p68 helicase polypeptide according to the invention can have the sequence as shown in FIG. 8. The RNA-helicase polypeptide can also be a p72 RNA-helicase polypeptide. The p72 RNA-helicase polypeptide can also have a sequence as in FIG. 10. The RNA-helicase polypeptide can be a p82 RNA polypeptide. The p82 RNA-helicase polypeptide can have a sequence as described for p82 in FIG. 10.

Furthermore, the document herein refers especially to a method for the treatment of a mammal (e.g. a human) having cancer (e.g. esophagus carcinoma, pancreatic carcinoma, stomach carcinoma, hepatocellular carcinoma, hepatoblastoma, cholangiocellular carcinoma). The method comprises the application of an inhibitor of an RNA-helicase polypeptide activity in a mammal. Preferably this is a helicase of the DEAD-box family. In an especially preferred embodiment, an inhibitor from the group of substances is given, which at least reduce the activity of the RNA-helicases p68, p72 and/or p 82. Advantageously, growth of the tumor is reduced by the treatment according to invention. It is especially advantageous when due to the treatment to a number of cancer cells in the patient (a mammal) is reduced as compared to the untreated patient. Within the spirit of the invention, it is already a successful treatment if the tumor shows a slower rate of growth of the growth of the tumor stagnates.

According to the invention, an inhibitor of the RNA-helicase is each substance which leads to a reduced activity of the RNA-helicases, preferably p68, p72 and/or p82 in the tumor cell. This definition includes inhibitors of gene expression as well as inhibitors of the enzymatic activity.

The mammal to be treated can be a human. The inhibitor can for example be the p68 RNA-helicase polypeptide which reduces activity in cancer cells (e.g. esophageal carcinoma, pancreatic carcinoma, stomach carcinoma, hepatocellular carcinoma, hepatoblastoma, cholangiocellular carcinoma but not mammary carcinoma) in the mammal. The inhibitor can for example be a siRNA-molecule, antisense oligonucleotide or ribozyme, which reduces the expression level of the p68 RNA-helicase polypeptide of the cancer cells (e.g. esophageal carcinoma, pancreatic carcinoma, stomach carcinoma, hepatocellular carcinoma, hepatoblastoma, cholangiocellular carcinoma).

The inhibitor according to the invention can also be an inhibitor of the p72 RNA-helicase polypeptide activity in the cancer cells (e.g. esophageal carcinoma, pancreatic carcinoma, stomach carcinoma, hepatocellular carcinoma, hepatoblastoma, cholangiocellular carcinoma but not mammary carcinoma). The inhibitor can be for example a siRNA-molecule, antisense oligonucleotide or ribozyme which can reduce the expression level of p72 RNA-helicase polypeptide in cancer cells (e.g. esophageal carcinoma, pancreatic carcinoma, stomach carcinoma, hepatocellular carcinoma, hepatoblastoma, cholangiocellular carcinoma).

The inhibitor can also reduce the p82RNA-helicase polypeptide activity in the cancer cells (e.g. esophageal carcinoma, pancreatic carcinoma, stomach carcinoma, hepatocellular carcinoma, hepatoblastoma, cholangiocellular carcinoma but not mammary carcinoma) in the mammal. The inhibitor can be for example a siRNA-molecule, antisense oligonucleotide or ribozyme which can reduce the expression level of p82 RNA-helicase polypeptide in cancer cells (e.g. esophageal carcinoma, pancreatic carcinoma, stomach carcinoma, hepatocellular carcinoma, hepatoblastoma, cholangiocellular carcinoma).

In a particularly advantageous embodiment of the invention, at least two inhibitors can be given in a therapeutically effective manner. Especially advantageous is the combination of an inhibitor of the p68 RNA-helicase or the p82 RNA-helicase. The inhibitors can be present in equal parts in the composition or they can be present in different parts.

If not specified otherwise, all technical and scientific terms that are used herein are the same as normally used in that field of the art of the invention as understood by those persons skilled in this art. All publications, patent applications or other references that are referred to herein are incorporated herein in their entirety.

The embodiments as described herein are only for the illustration of the invention and are not understood to be limiting in any way.

Other features and advantages of the invention are shown from the following detailed description and from the claims.

### DESCRIPTION OF THE FIGURES

FIG. 1

Characterization of the anti-68 (555-576) antibody. (A) Anti p68 Western blot of entire cell lysate from the human breast cancer cell line (MDA-MB-231 and Hs578T. (B) Peptide-competition. A peptide which comprises the amino acids 555 to 576 of the p68 RNA-helicase, suppresses the recognition of endogenous p68 RNA-helicase via the anti-68-(555-576) antibody in a Western blot with Hs578T cell extract, whereas a non-related peptide does not. (C) The p68 RNA-helicase expression in MDA-MB-231 or Hs578T breast cancer cells was tested through with an immunological stain by using the anti-p68-(555-576) antibody. DNA was stained with a Hoechst dye.

FIG. 2

Immunohistochemical analysis of the p68 expression (white fluorescence signal) in liver tissue with use of the α-p68-(555-576)-antibody.

(A) normal tissue. (B, C) hepatocellular carcinoma.

FIG. 3

Immunohistochemical analysis of p68 expression (white, fluorescence signal) in tissue from the human gall duct by using the α-p68-(555-576)-antibody.

(A) normal tissue (gall duct). (B, C) cholangio-carcinoma.

FIG. 4

Immunohistochemical analysis of p68 expression (white, fluorescence signal) in pancreas tissue by using the α-p68-(555-576)-antibody

(A) normal tissue. (B, C) ductal adenocarcinoma.

FIG. 5

Immunohistochemical analysis of p68 expression (white, fluorescence signal) in stomach tissue by using the α-p68-(555-576)-antibody.

(A) normal tissue (B, C) adenocarcinoma.

FIG. 6

Immunohistochemical analysis of p68 expression (white, fluorescence signal) in tissue from the esophagus by using the α-p68-(555-576)-antibody.

(A) normal tissue (B, C) adenocarcinoma.

FIG. 7

Nucleic acid sequence coding for human p68 RNA-helicase polypeptide which (NM_{—}004396 coding area 171-2015).

FIG. 8

Amino acid sequence of the human RNA-helicase polypeptide.

FIG. 9

Nucleic acid sequence coding for human p82 RNA-helicase polypeptide or for p72-Isofrom (NM_{—}006385, coding area for p82: 75-2264, coding area for p72: 312-2264).

FIG. 10

Amino acid sequence of human p72 respectively p82 RNA-helicase polypeptide (the amino acid sequence) for p82 starts with amino acid 1 and ends with amino acid 729; the amino acid sequence for p72 starts at amino acid residue 80 and ends with amino acid 729 (NP_{—}006377).

FIG. 11

(A) Human RKO colon cancer cells were infected three times with a mixture of two retro viruses (right track) respectively control viruses (left track). Retrovirus 1 encodes the expression of a shRNA (small hairpin RNA) directed against human p68, retrovirus 2 a shRNA, which is directed against a human p72. After 48 hours protein extracts were made from the cell and separate by gel electrophoresis. In the illustrated Western blots to these protein extracts a distinct down regulation of p68 and p72/p82 protein is shown, while the control protein actin remains uninfluenced. Cyclin D1 and c-Myc were likewise down-regulated in the presence of p68+p72/p82 shRNA. In addition, an up regulation of the activated form of the caspase 3 can be seen.

(B) RKO cells as in A) were seeded with 25% confluence and two days later examined under the phase contrast microscope. The control cells form an almost confluent cell lawn, while the cell infected with p69+p72/p82 shRNA appear hardly increased and numerously rounded off.

(C) and (D) 3.5×10⁶ RKO cells were treated like under (A) and injected in the left flank of naked mice. After 32 days, the resulting tumors were isolated. (C) Shows representatively a tumor each from control and cells treated with p68+p72'p82 shRNA. (D) Shows the statistical interpretation of the experiments regarding the investigated tumor mass with eight mice each in both groups. The average tumor mass for the cell treated with p68+p72/p82 shRNA is distinctly reduced as compared to the control group.

FIG. 12

Immunoblot-analysis of the p68 RNA-helicase expression level in human carcinoma cell lines (A) cells of the human cell line Hep-G2 of the hepatocellular carcinoma were lysated and a portion of the lysate, relative to the protein content thereof, separated by means of a SDS-polyacrylamid gel electrophoresis (SDS-PAGE) and blotted on an ECL Western blot membrane. The test was carried out with the antibody against p68 α p68-1583 primary antibody followed by a peroxidase coupled secondary antibody and a following ECL reaction. The determination of the elevated p68 expression is done by comparison with the non-tumorgenic permanent endothelial human cell line Hs BST (cf. 12G).

(B) Cells of the human cell line of the pancreas carcinoma ASPC1 were treated like under (A). The elevated expression of p68 is detected through comparison with 12G.

(C) Cells of the human cell line PaTU of the pancreas carcinoma were treated like under (A). The elevated expression of p68 is detected through comparison with 12G.

(D) Cells of the human cell line KYSE 70 of the weakly differentiated esophagus carcinoma were treated like under (A). The elevated expression of p68 is detected through comparison with 12G.

(E) Cells of the human cell line KYSE 140 of a moderately differentiated esophagus carcinoma were treated like under (A). The elevated expression of p68 is detected through comparison with 12G.

(F) Cells of the human cell line KYSE 510 of well differentiated esophagus carcinoma were treated like under (A). The elevated expression of p68 is detected through comparison with 12G.

(G) Cells of the non-tumorgenic endothelial cell line HsBST were treated like under (A). The detection of p68 shows only a weak expression.

(H) Cells of the human cell line MCF7 of the mammary carcinoma were treated as a positive control like under (A). The elevated expression of p68 is tested by comparison with 12G and corresponds with Janknecht, 2006 (WO 2006/002053 A2).

FIG. 13

Immunocytochemical test of the elevated expression of p68 in human tumor cells (A) cells of the human cell line Hep-G2 of the hepatocellular carcinoma were fixated and after saturation of the unspecific protein binding sites with BSA visualized in the fluorescence microscope first with the primary antibody α-p68-1583 and further with a secondary antibody Alexa 488 which had been fluorescence marked. An elevated p68 expression was determined through comparison with the positive control of the human breast cancer cell line MCF7 documented as being overexpressive.

(B) Cells of the human cell line ASPC1 of the pancreas carcinoma were treated like under (A) and the overexpression of p68 determined in analog manner.

(C) Cells of the human cell line PaTU of the pancreas carcinoma were treated like under (A) and the overexpression of p68 was detected in analog manner.

(D) Cells of the human cell line KYSE 70 of the weakly differentiated esophagus carcinoma were treated like under (A) and the overexpression of p68 was detected in analog manner.

(E) Cells of the human cell line KYSE 140 of a moderately differentiated invasively growing esophagus carcinoma were treated like under (A) and the overexpression of p68 was detected in analog manner.

(F) Cells of the human cell line KYSE 510 of well differentiated esophagus carcinoma were treated like under (A) and the overexpression of p68 was detected in analog manner.

(H) Cells of the human cell line MCF7 of the mammary carcinoma were treated as a positive control like under (A). The elevated expression of p68 is tested by comparison with 12G and corresponds with Janknecht, 2006 (WO 2006/002053 A2).

### DETAILED DESCRIPTION

The present invention comprises methods and materials for the diagnosis and treatment of rare cancers such as hepatoblastoma, hepatocellular, cholangio-, pancreas-, esophagus- and stomach cancer in mammals (e.g. humans, dogs, cats, horses, cows, goats, pigs and rodents).

According to the invention for the purpose of diagnosis known methods for the quantitative and qualitative testing of an RNA-helicase in the afflicted tissue are applied. Especially certain methods are used to determine whether a patient probe contains an elevated level of RNA-helicase (example p68, p72 and/or p82 RNA-helicase) polypeptide. As herein stated, a patient can be classified as “having cancer” or at least classified as “suspected of having cancer” if the level of RNA-helicase for example of a RNA-helicase of the DEAD box family such as p68, p72 and/or p82 RNA-helicase polypeptide in a probe is shown to be elevated.

The level p68, p72 and/or p82 helicase polypeptide can be determined by measuring of each p68, p72, p82 RNA-helicase polypeptide inclusive of native and mutated but not limited to p68, p72, p82 RNA-helicase polypeptide. Examples for the p68 RNA-helicase polypeptide comprise without limitation, human p68 RNA-helicase polypeptide (e.g. GenBanK® access number NP_{—}006377, FIG. 10), horse-p72 RNA-helicase polypeptide, rabbit-p72 RNA-helicase polypeptide and mouse-p72 RNA-helicase polypeptide.

Examples for p82 RNA-helicase polypeptide comprise without being limited to those, humane p82 RNA-helicase polypeptide (e.g. GenBanK® access number NP_{—}006377, FIG. 10), horse-p82 RNA-helicase polypeptide, rabbit-p82 RNA-helicase polypeptide and mouse p82 RNA-helicase polypeptide.

The term “elevated level” as used here refers to a level of RNA-helicase (e.g. p68, p72 and/or p82 RNA-helicase) polypeptide. The term “reference level” as used here with respect to a RNA-helicase (P68, p72 and/or p82 RNA-helicase) polypeptide level as typically not expressed in cancerous/healthy mammals (e.g. humans). The reference level of a p68 RNA-helicase polypeptide can be for example the average level in p68 RNA-helicase polypeptide, which is contained in probes from 50 randomly chosen healthy mammals of the receptive species.

It is advantageous when comparable probes can be utilized whether or not the observed level is an elevated level or not. For example the average p68, p72 and/or p82 RNA-helicase polypeptide level in a healthy normal tissue probe of a certain organ from the same randomly chosen group of the same species can contain a certain number X units/g normal tissue, while that average respective level of p68, p72 and/or p82 RNA-helicase polypeptide in the tumorous tissue of the same organ from the same randomly chosen group of mammals deviates, namely for example shows a comparatively smaller amount of Y units/g tumor tissue.

The reference level for the RNA-helicase polypeptide level in the tumor tissue probe from a certain organ would accordingly be the level of the RNA-helicase polypeptide in the normal tissue probe of the respective organ. For tumor probes, which are taken from another tissue, the determination whether its RNA-helicase polypeptide level is elevated or not, should possibly be done by referring to the level of RNA-helicase polypeptide in the associated normal tissue. Associated In this connection is referred to that the normal tissue is taken from the same organ (possibly from another patient) from which the tumor is derived; the determination of the reference level would then be from this normal tissue. Accordingly, it is possible that the reference level is the result of a determination form the non-diseased tissue portion of the identical organ or correspondingly from the non-diseased tissue of another patient. In each case, the reference probe is to be used for diagnosis.

At least however, the determination of the RNA-helicase polypeptide level in the tumor probe must be carried out based on a reference level which was detected in the normal tissue and which is from the same tissue type as the tumor probe, even when the normal tissue probe is derived form another individual. For example, a liver tumor probe should be compared with a liver normal tissue from the same patient; if this is not feasible, alternatively a comparison with the normal liver tissue from the liver of another patient should be utilized.

The level which points to the pathological condition has no absolute value. Rather, the elevated level for the RNA-helicase polypeptide (ex. P68-, p72-, or p82 RNA-helicase) polypeptide can basically take on any level, provided the level is higher than the corresponding reference level for the RNA-helicase (ex. P68-, p72-, or p82 RNA-helicase) polypeptide. For example, an elevated level for an RNA-helicase polypeptide 0.1, 0.5, 1, 2, 3, 4, 5, 6, 7, 8, 9, 10, 15 20 fold or further manifold higher than the reference level for a RNA-helicase polypeptide in the reference probe. Furthermore, the reference level can have any value and may even be zero for the p68 RNA-helicase polypeptide. In that case, any level greater than zero for the 68 RNA-helicase polypeptide would be an elevated level.

Basically, any known method can be used in order to determine the level of the RNA-helicase polypeptide (e.g. p68, p72 and/or p82 RNA-helicase) polypeptide which is present in the probe. For example anti p68 RNA-helicase polypeptide antibodies can be used to investigate the expression level of the p68 RNA-helicase polypeptide in a probe.

In some embodiments, the level of a RNA-helicase polypeptide in a probe can be determined by means of protein chemical detection methods such as Western blot or immunochemical techniques.

Another method which may be utilized to examining a p68, p72 and/or p82 RNA-helicase polypeptide in a probe can be functional. For example, an ATPase assay can be used to determine whether or not a tissue probe contains an elevated level of p68 RNA-helicase polypeptide.

The level of an RNA-helicase polypeptide (e.g. p68, p72 and/or p82 RNA-helicase) polypeptide which is present in a probe can also be determined by means of measuring a level of an mRNA which codes for a RNA-helicase (e.g. p68, p72 and/or p82 RNA-helicase) polypeptide. Each method can be used in order to measure the level of an RNA which codes for a RNA-helicase (e.g. p68, p72 and/or p82 RNA-helicase) polypeptide, including without being limited thereto, PCR based methods. For example RT-PCR with oligo primers can be used, which were designed for the amplification of nucleic acid (e.g. RNA), which codes for a p68, p72 and/or p82 RNA-helicase polypeptide.

Any method can be used to identify primers that are capable to amplify a nucleic acid or nucleic acids, which encodes a RNA-helicase polypeptide. For example a computer algorithm can be used to scan a data bank (e.g. Genbank®) for p68 RNA-helicase nucleic acid.

Any method can be used in order to analyze the amplified products. For example amplified products, corresponding to p68, p72 and/or p82 RNA-helicase mRNA can be separated by gel electrophoresis and the level of p68, p72 and/or p82 RNA-helicase specific product determined by densitometry. Alternatively, the level of the p68, p72 and/or p82 RNA-helicase specific product can be determined via quantitative RT-PCR by using fluorescence signal molecules or dyes.

All types of probes including a tissue probe can be used in order to determine the level of a RNA-helicase (p68, p72 and/or p82 RNA-helicase) polypeptide. Furthermore any method can be sued in order to obtain the probe. For example the tissue probe can be obtained by a biopsy Once obtained, a probe, prior to measuring the level of a RNA-helicase (p68, p72 and/or p82 RNA-helicase) polypeptide can be treated. For example a tissue probe can be so treated, that mRNA is obtained. Once received, the mRNA can be analyzed, in order to determine the level of p68, p72 and/or p82 RNA-helicase mRNA. In a further example, a tissue probe can be treated so that a cell lysate is obtained. Once the cell lysate is available, it can be analyzed by using anti-RNA-helicase polypeptide antibodies (e.g. p68, p72 and/or p82 RNA-helicase polypeptide antibodies) in order to determine the level of RNA-helicase (p68, p72 and/or p82 RNA-helicase polypeptide) in the probe.

The present invention provides a method to support medical and research personnel in the testing whether or not a mammal has cancer. Medical personnel may be for example physicians, nurses, medical technical lab personnel or pharmacists. Research trained personnel can include for example research group leaders, technical assistants, PhD scientists and doctoral students.

The diagnostic method according to the present invention can be advantageously carried out with an antibody directed against the RNA-helicase polypeptide, preferably against the p68 RNA-helicase polypeptide directed antibody, the coupling of which is detected by an immunological method such as for example an immunohistochemical staining or by an immuno blot.

### p68 as Therapeutic Target

The present description provides a method for the treatment of mammals, especially humans, who suffer from a cancer disease (e.g. mammals suffering from a hepatoblastoma, a hepatocellular-, a cholangio-, a pancreas-, a stomach- or an esophagus carcinoma). The method according to the invention can for example be used for treating a mammal suffering from cancer in such a manner that the number of cancer cells will get reduced (e.g. a 10, 20, 30, 40, 50, 60, 70, 80, 90, or 100% reduction.) or that the growth of the tumor is being slowed down or stagnates. The method according to the invention is applicable to all type of tumors. It is however not claimed for breast cancer.

According to the method of the invention, at least one compound is administered to the mammal suffering from cancer, whereby this compound reduces the level of RNA-helicase (e.g. p68, p72 and/or p82 RNA-helicase) polypeptide activity in the cancer cells of the mammal. Any type of compound can be used in order to reduce the level of RNA-helicase (e.g. p68, p72 and/or p82 RNA-helicase) polypeptide activity in the cancer cells of the mammal.

For example any inhibitor of a p68, p72 and/or p82 RNA-helicase polypeptide activity (e.g. ATPase inhibitors) can be used. In a preferred embodiment antisense molecules, shRNA (small hairpin RNA). siRNA molecules, RNAi constructs and/or RNA oligos can be utilized in order to reduce the level of p68, p72 and/or p82 RNA-helicase polypeptide activity in the cancer cells of a mammal, where the level of p68, p72 and/or p82 RNA-helicase polypeptide expression in the cells are reduced.

In some cases one or more compounds can be given to a mammal suffering from cancer in such a manner that the level of more than one RNA-helicase activity in the cancer cells of the mammal is being reduced. For example, a single compound can be administered in order to reduce the level of p68, p72 and/or p82 RNA-helicase polypeptide activity. In another example, two compounds can be administered to the mammal suffering from cancer: one compound (e.g. an siRNA molecule), which is directed against the expression of the p68 RNA-helicase polypeptide), which can reduce the level of p68 RNA-helicase polypeptide activity and a further compound (e.g. a siRNA molecule), directed against the expression of p72 and also of p82 RNA-helicase polypeptide), which can reduce the level of p72 as well as the level of P82 RNA-helicase polypeptide activity.

The invention is described in more detail in the following embodiments, which should not be taken to limit the scope of the invention as claimed.

### Embodiments

### 1. Immunohistochemical Test of 68 RNA-Helicase

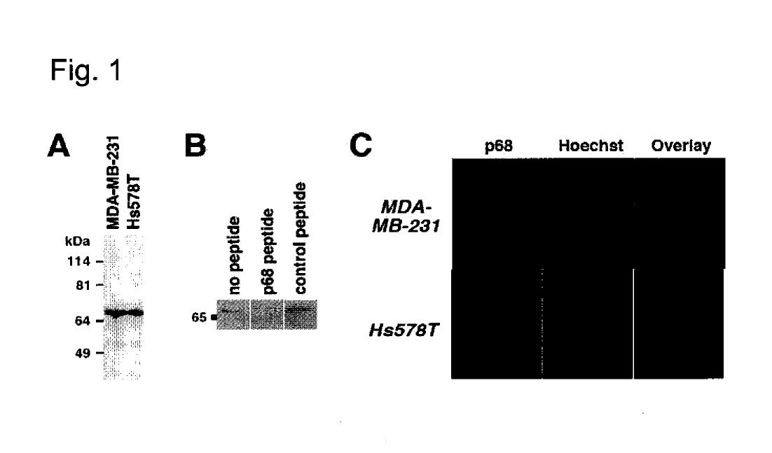

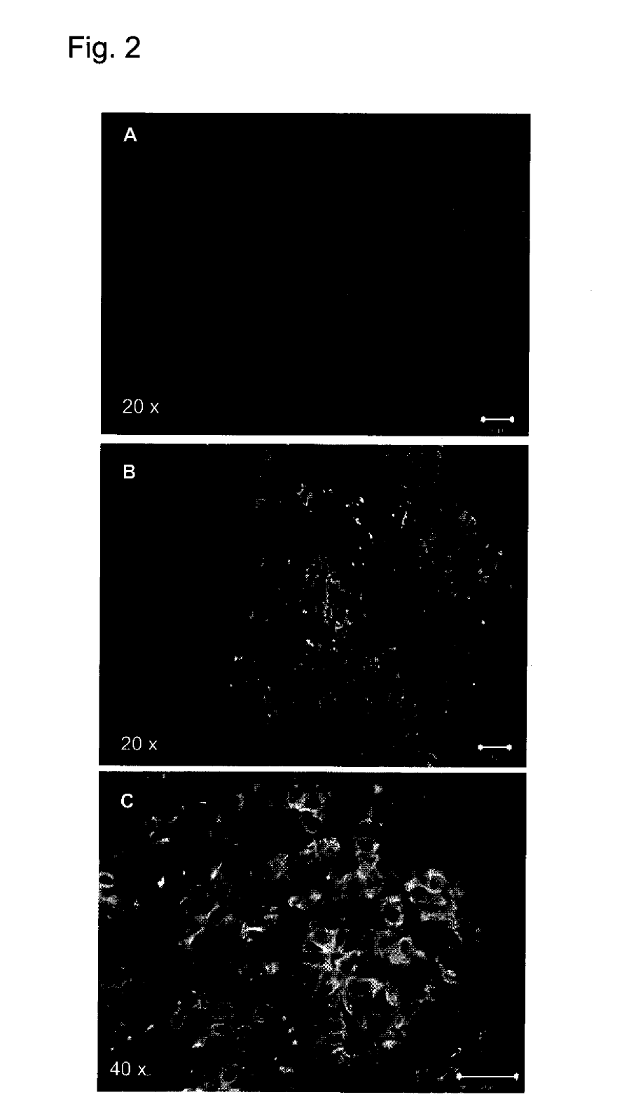

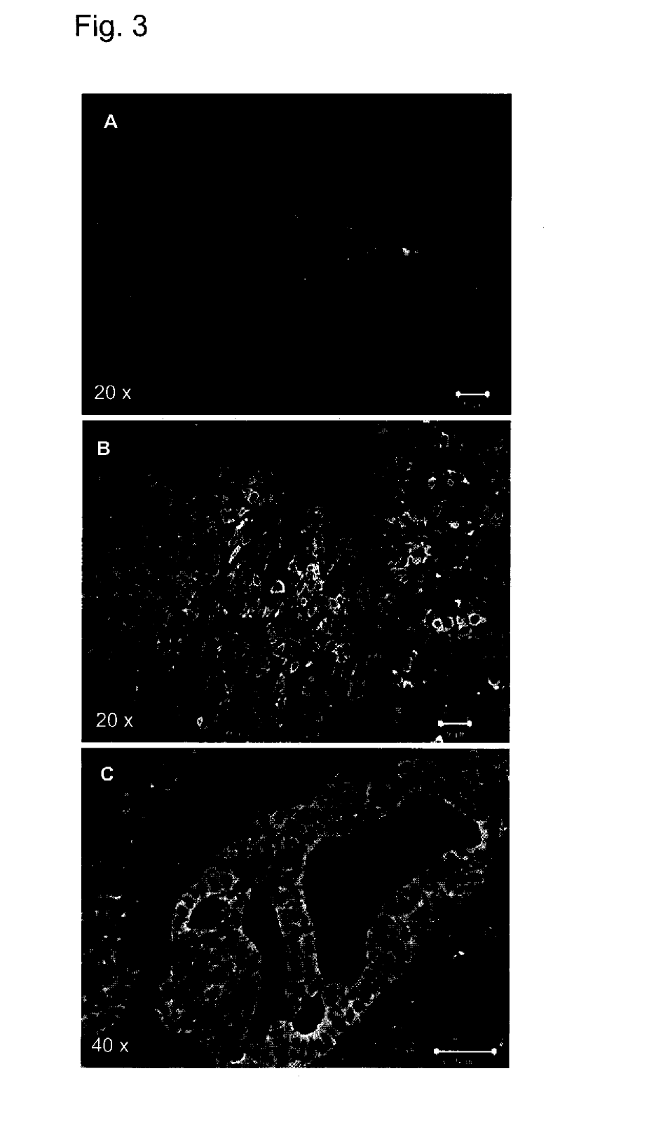

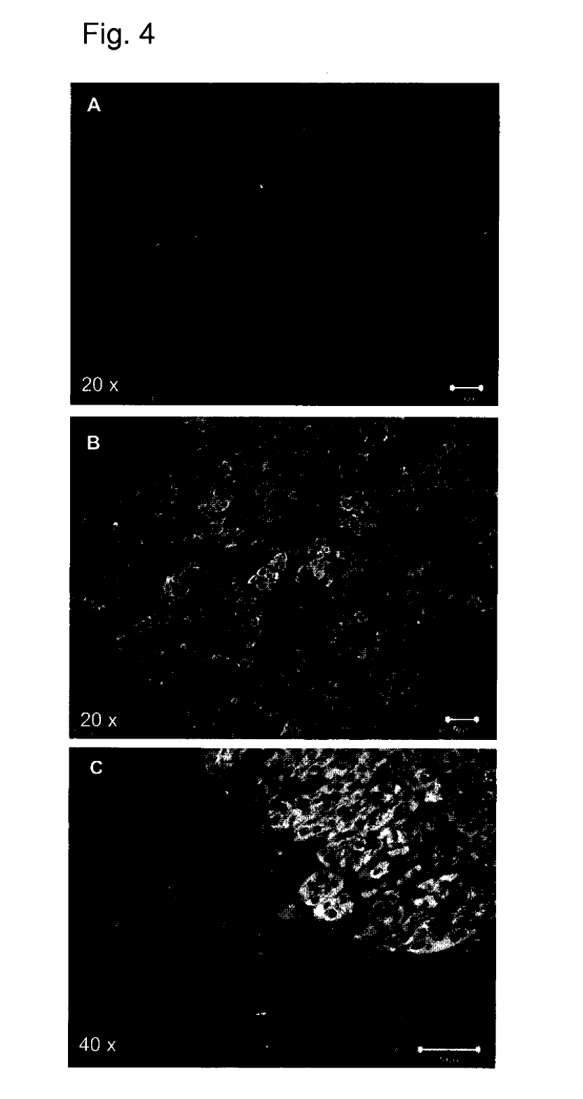

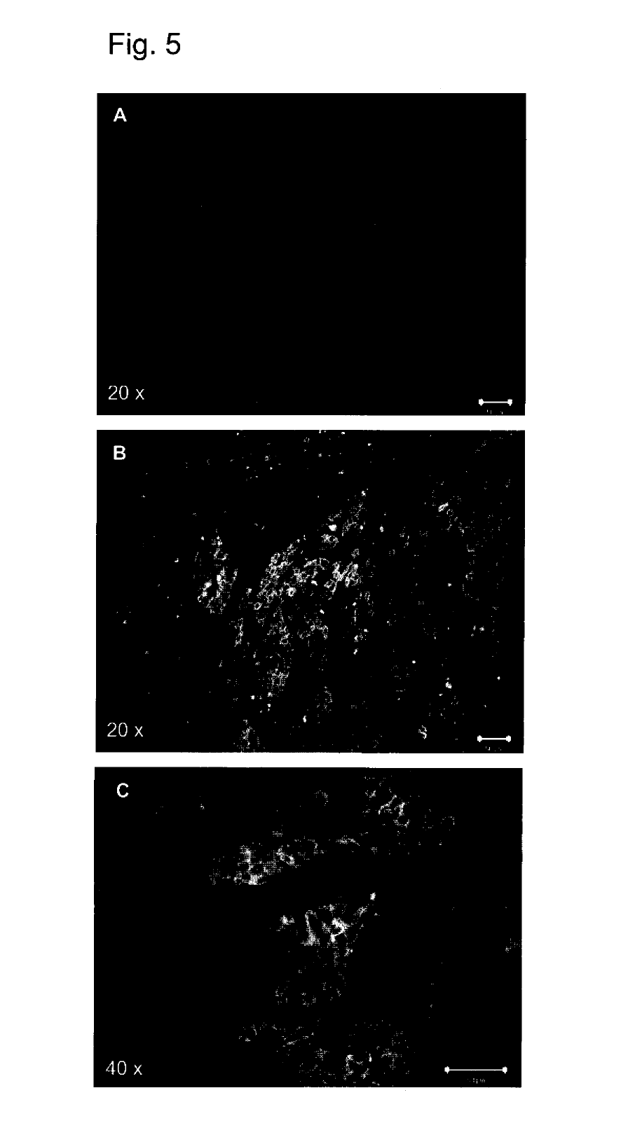

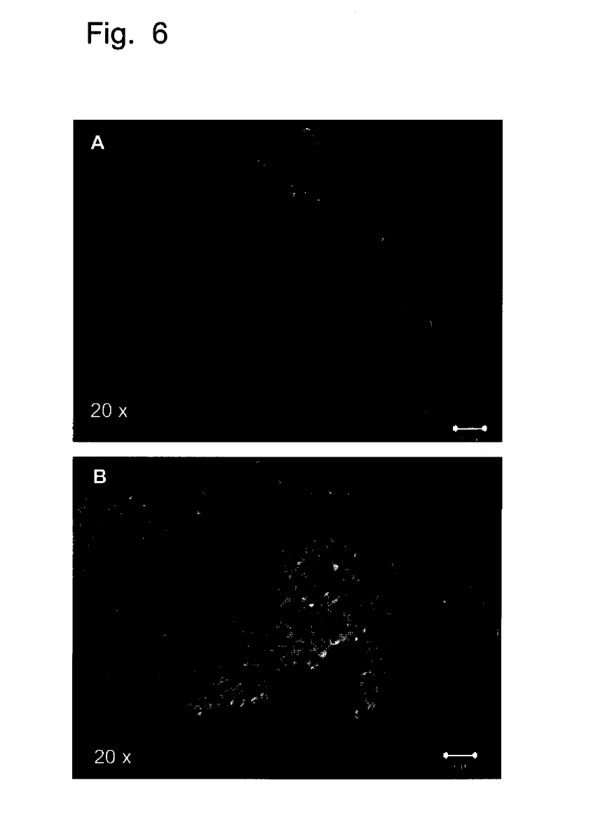

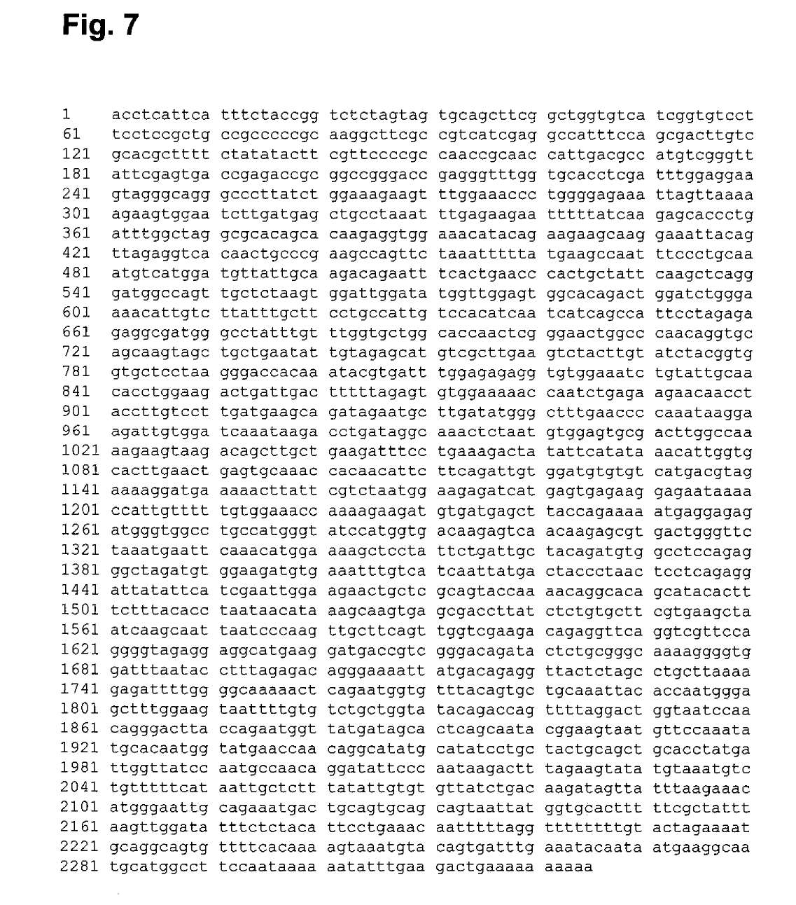

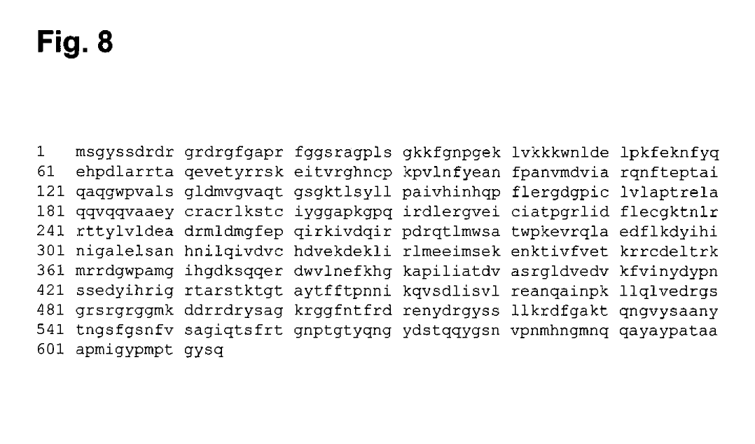

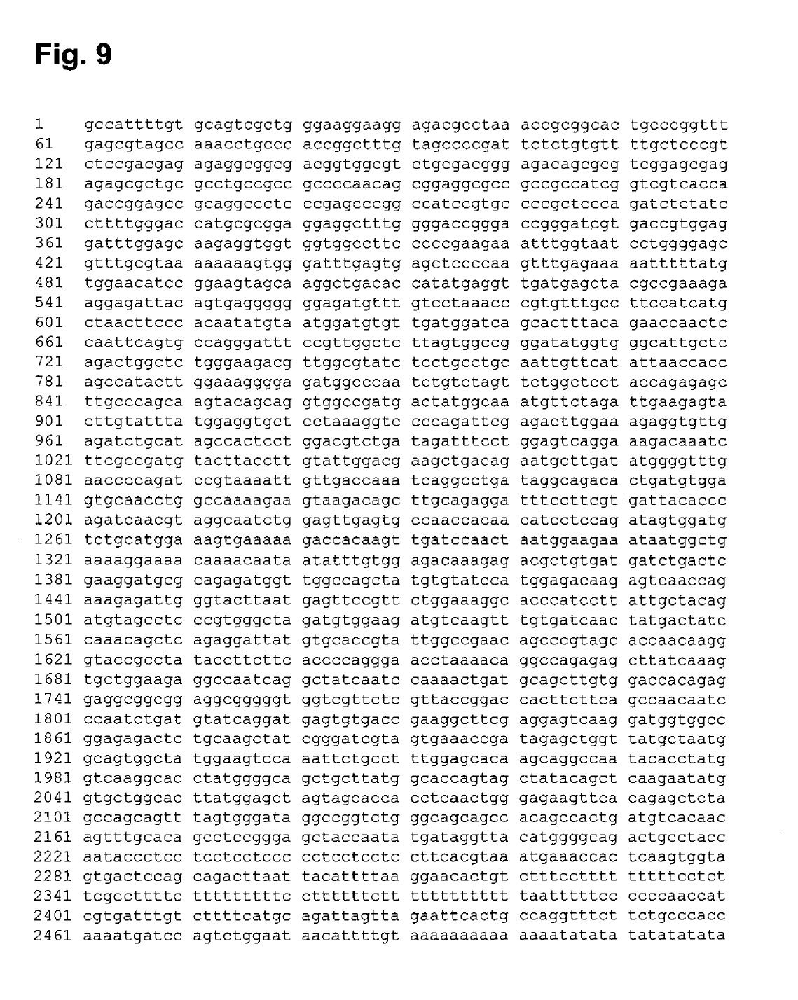

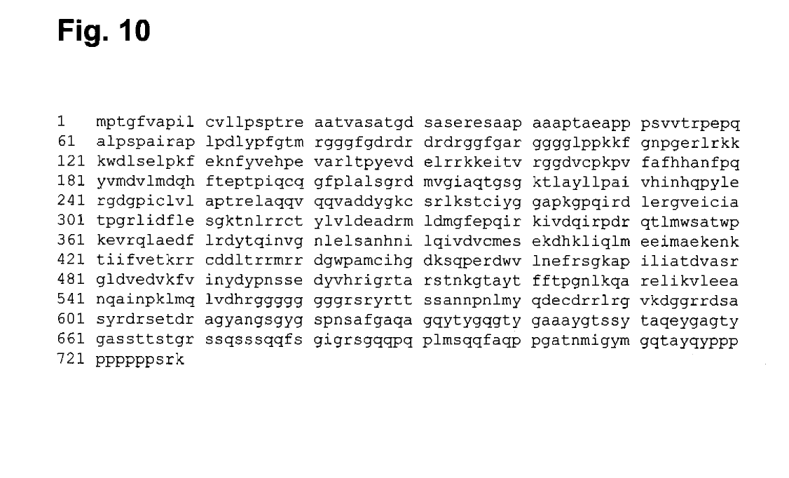

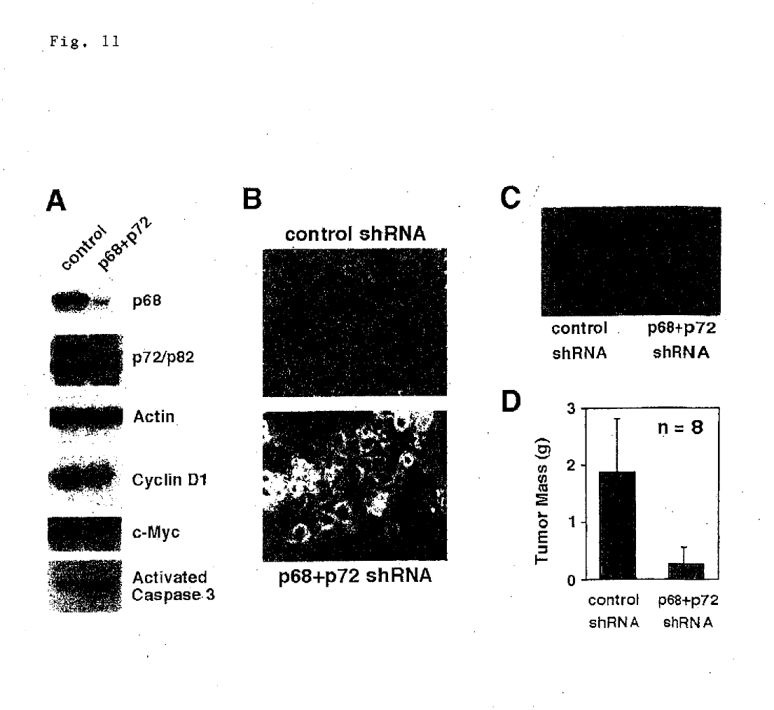

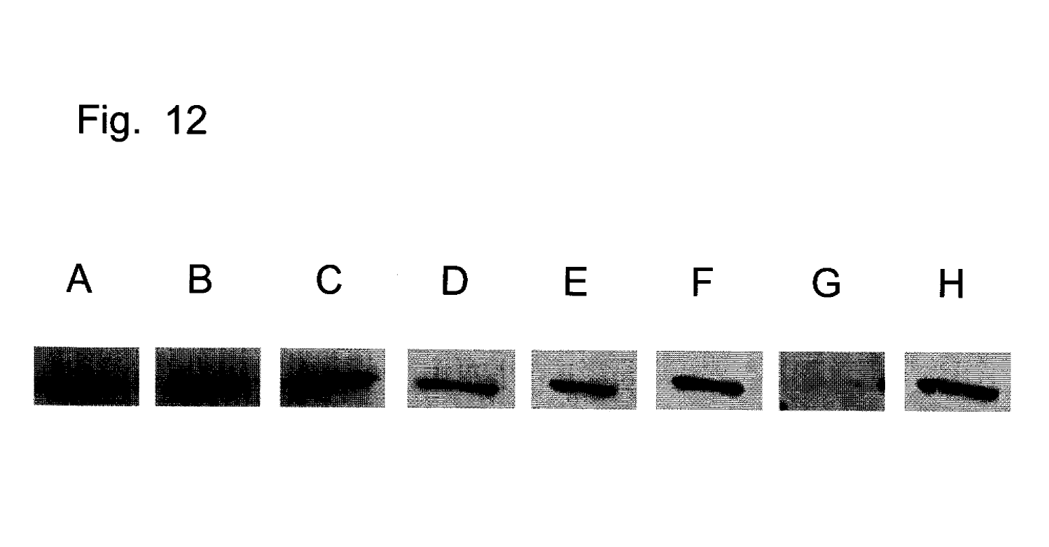

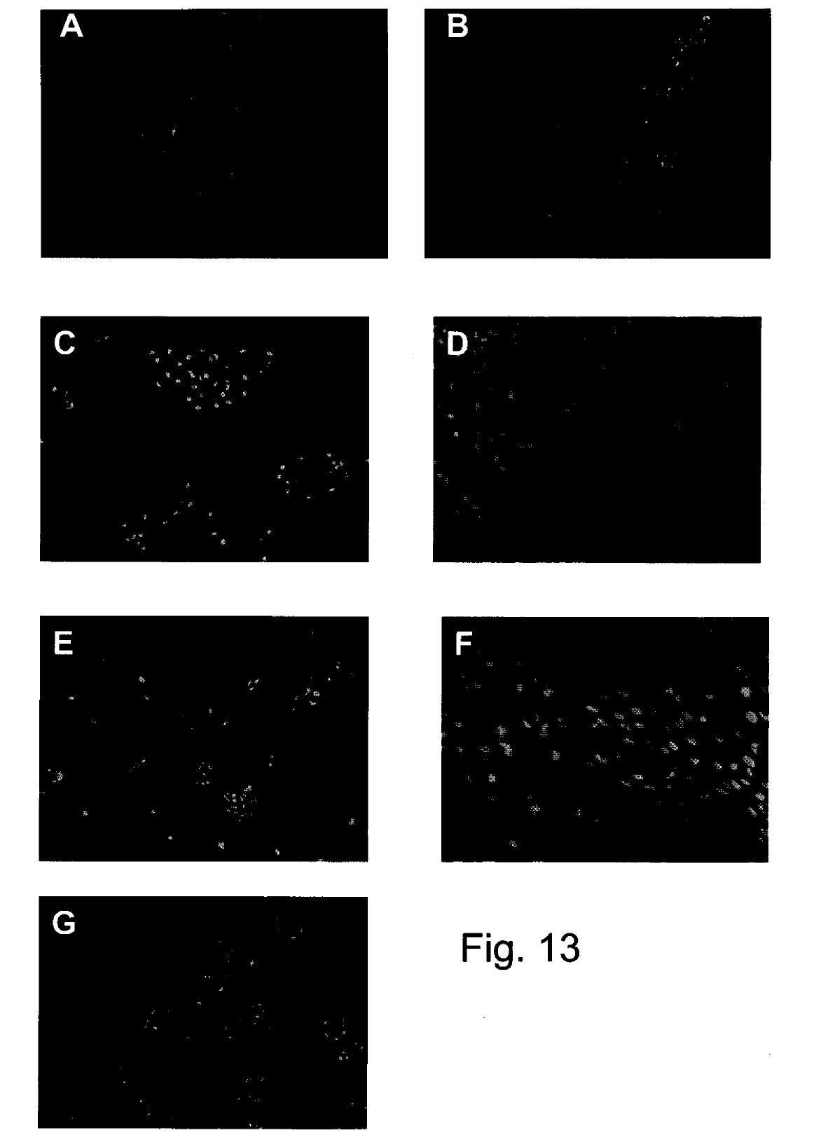

A polyclonal anti-p68-antibody was developed from rabbits in order to immunohistochemically stain the tissue probes. The antibody is directed against the amino acids 555 to 576 and was affinity purified (α-p68-(555-576)) against the respective oligopeptide. This antibody recognizes specific endogenous p68 RNA-helicase (cf. FIG. 1) [Rossow and Janknecht] and was used in the present example for an immunohistochemical staining of probes embedded in tissue probes. In the same manner two further antibodies were obtained from rabbits, one of which is directed against α-p68-1583, against amino acids 501 to 524 and others, α-p68-1581 against amino acid 4 to 25.

For immunohistochemical staining tissue microarrays were used, which next to normal tissue probes comprise numerous tumor probes of a certain tumor type, whereby the normal and abnormal tissue probes are in part from the same patient.

After de-paraffination and rehydrating of the approximately 10 μm thick tissue cut, which was followed by a heat induced “antigen retrieval”, non-specific protein binding sites were saturated with a BSA (bovine serum albumin) solution. P68 in the tissue was detected by using the anti-p68 primary antibody α-p68-(555-576) and an Alexa fluor 488 secondary antibody conjugate (goat) and evaluated after embedding in Moviol/Dabco with the aid of a fluorescence microscope (Axiovert 200, Zeiss). When balancing the fluorescent signals of prior documented auto fluorescence or unspecific background binding of the secondary antibody, a precise determination of the specific p68 RNA-helicase signal. With this method, small expression differences can also be demonstrated by a good signal background ratio.

In addition, a further evaluation was made of the results with the antibody α-p68-1583, whereby the results with the antibody α-p68-(555-576) were confirmed. For further investigations the p68 antibody α-p68-1581 can also be used.

Further investigated were tissue microarrays with probes from hepatocellular carcinomas, cholangio-carcinomas, pancreas carcinomas, stomach carcinomas and esophagus carcinomas.

### 1.1 Definition of Terms for the Evaluation

Negative: a probe is classified as “negative”, when localization and intensity of the p68 signal after the immuno fluorescence staining corresponds to the level of the auto fluorescence of the unspecified background.

Moderate: probes are classified as “moderate”, if they show a weak to moderate elevation of the p68 signal in comparison to the auto fluorescence and/or to the unspecified background.

Strong: a probe is classified as strong, when a distinct elevation of the p68 signal is detected as compared to the auto fluorescence and/or the unspecific background staining in neoplastic tissue; even when the signal is limited to single structures/cell groups within the tissue probe. When necessary, the non-neoplastic probes that are showing a corresponding expression level are also being classified.

Very Strong: this classification is used for probes with an extremely conspicuous and signal intense staining pattern.

### 1.2 Hepatocellular Carcinoma Microarray

As described, p68 was stained in a hepatocellular carcinoma microarray and the expression level of the normal- and tumor tissue compared and evaluated. A high degree of auto fluorescence was already shown before the specific immunohistochemical staining. A detected p68 signal was distinguishable to a distinct degree (FIG. 2).

As shown in Table 1, when balancing the signal for the auto fluorescence and the normal tissue in 37% of the tumor tissue probes, the p68 signal was classified as strong, which corresponds to a distinctly elevated expression level of the protein. In 9% (“negative”) of the cases, none or only a very weak expression of p68 tissue was detected after compensation for auto fluorescence and specific background, in 54% the elevation of the p68 level was declared as moderate (“moderate”).

TABLE 1
Evaluation of the Hepatocellular Carcinoma Microarray

| | For neoplastic probes/ |
|---|---|
| p68 Signal | total number of neopl. probes |
| negative (0) | 3/35 (9%) |
| moderate (1) | 19/35 (54%) |
| strong (2) | 13/35 (37%) |
| very strong (3) | 0 |

### 1.3 Cholangio Carcinoma Microarray

The p68 RNA-helicase was as described stained in the cholangio carcinoma microarray and the expression level between the normal- and tumor tissue compared and evaluated. A high degree of auto fluorescence was already shown before the specific immunospecific staining. A detected p68 signal differed distinctly (FIG. 3).

As can be seen from Table 2, the p68 signal, when compensated for auto fluorescence and normal tissue was classified in 37% of the tumor tissue probes as strong or very strong, which corresponds to an elevated expression level of the protein. In 11% (“negative”) the cases, a very weak or no expression of p68 tissue was detected when compensated for auto fluorescence and unspecific background, in 52% the elevation of the p68 level was to be classified as moderate (“moderate”).

TABLE 2
Evaluation of Cholangio Carcinoma Microarray

| | For neoplastic probes/ |
|---|---|
| p68 Signal | Total number of neopl. probes |
| negative (0) | 5/46 (11%) |
| moderate (1) | 24/46 (52%) |
| strong (2) | 16/46 (35%) |
| very strong (3) | 1/46 (2%) |

### 1.4 Pancreas Carcinoma Microarray

P68, as described, stained in the pancreas tissue probes and the expression level of the normal- and tumor tissue compared and evaluated. It was shown thereby that already before the specific immunohistochemical staining there was weak auto fluorescence. A detected p68 signal thereby differed distinctly (FIG. 4).

As seen from table 3, the p68 signal when balanced for auto fluorescence and normal tissue is classified in 48% of the tumor probes as strong or very strong, corresponding to an elevated expression level of the protein. In only 3% (“negative”) of the cases none of only a very weak expression of p68 in the tissue after compensating for auto fluorescence and unspecific background, in 49% the elevation of the p68 level was classified as moderate (“moderate”).

TABLE 3
Evaluation of the Pancreas Carcinoma Microarray

| | For neoplastic probes/ |
|---|---|
| p68 Signal | Total number of neopl. Probes |
| negative (0) | 1/33 (3%) |
| moderate (1) | 16/33 (49%) |
| strong (2) | 15/33 (45%) |
| very strong | 1/33 (3%) |

### 1.5 Stomach Carcinoma Microarray

P68 was, as described, stained in the stomach carcinoma microarray and the expression level compensated and evaluated with the normal-tumor tissue. A weak auto fluorescence was shown thus even before the specific immunohistochemical staining. A detected p68 signal differed however considerably (FIG. 5).

As seen in Table 4, the p698 signal, when balanced for auto fluorescence and normal tissue, in 24% of the tumor probes, was classified as strong which corresponds to a distinctly elevated level of the protein. In 14% (“negative”) of the cases none or only a very weak expression of p68 was detected in the tissue after compensating for auto fluorescence and unspecified background, in 62% of the cases the elation of the p68 level could be classified as moderate (“moderate”).

TABLE 4
Evaluation of Stomach Carcinoma Microarray

| | For neoplastic probes/ |
|---|---|
| p68 signal | Total number of neopl. probes |
| negative (0) | 7/50 (14%) |
| moderate (1) | 31/50 (62%) |
| strong (2) | 12/50 (24%) |
| very strong (3) | 0 |

### 1.6 Esophagus Carcinoma Microarray

P68 was, as described stained in the esophagus carcinoma microarray and the expression level compared and evaluated with the normal-tumor tissue. A high degree of auto fluorescence and unspecified coupling to the secondary antibody was shown already prior to the specific immunohistochemical staining. A detected p68 signal could be distinctly differentiated therefrom (FIG. 6).

A seen from Table 5, the p698 signal, when compensated for auto fluorescence and normal tissue, in 25% of the tumor probes, was classified as strong which corresponds to a distinctly elevated level of the protein. In 8% (“negative”) of the cases none or only a very weak expression of p68 was detected in the tissue after compensating for auto fluorescence and unspecified background, in 67% of the cases the elevation of the p68 level could be classified as moderate (“moderate”).

TABLE 5
Evaluation of Esophagus Carcinoma Microarray

| | For neoplastic probes/ |
|---|---|
| p68 signal | Total number of neopl. probes |
| negative (0) | 3/40 (8%) |
| moderate (1) | 37/40 (67%) |
| strong (2) | 10/40 (25%) |
| very strong (3) | 0 |

### 2. Test of RNA-Helicase Polypeptide in Tissue Extracts

The following experiments serve as detection of the basic mechanism of the p68 polypeptide overexpression in tumors.

2.1

The strength of expression of the p68 RNA-helicase polypeptide in hepatocellular, cholangio-, pancreas-, stomach- and esophagus carcinoma is determined by an immuno blot method. For that the entire protein from the carcinogenic and the corresponding normal tissue (non-diseased tissue) of the corresponding tumor type is isolated (commercially available e.g. from BioCat) and separated according to their molecular weight by means of SDS-polyacrylamid gel electrophoresis (SDS-PAGE). The test of p68 follows thereafter in an immuno blot with a polyclonal antibody which is directed specifically against the p68 protein and with a secondary antibody conjugated with peroxidase. The expression strength of p68 can then be read from the strength of the test reaction (enhanced chemiluminescence System, ECL system) and correlated to the amount of the p68 present in the examined tissue. A comparison with the expression level in the normal tissue shows an overexpression of the carcinogenic tissue.

2.2

The expression strength of the p68 RNA-helicase polypeptide in hepatocellular, cholangio-, pancreas-, stomach-, and esophagus carcinoma is determined by means of an “immunoassay” (e.g. the ELISA, enzyme linked immunosorbent assay). For this purpose, all proteins from the carcinogenic and corresponding normal tissue (non-diseased tissue) of the corresponding tumor types are isolated. (commercially available e.g. from BioCat) and immobilized in a “multiwell ELISA plate”. After saturating of protein binding sites with BSA solution, the test for RNA-helicase follows by means of a specific polyclonal antibody which is specific for the p68 protein and an enzyme (e.g. alkaline phosphatase or peroxidase) or fluorescent dye conjugated secondary antibody. The expression strength of p68 is then selected from the enzymatic test reaction (a substrate conversion leads to a colored product) or the fluorescence intensity in an ELISA reader and correlates with the amount of p68 present in the examined probe. A comparison with the expression level in normal tissue shows an overexpression in the carcinogenic tissue.

These immunoassays are especially advantageous since they can be automated for mass analyses.

2.3

The following experiment serves a speedy, comprehensive and accurate identification of tumor specific expression changes of single genes. In short: the cancer profiling array (BD Clontech) contains an expression profile from 19 tumor types and corresponding normal tissue, which are represented by 154 probe pairs, whereby most of the tumors are represented by at least 10 patient probes. The RNA isolated from the tissue probes was transcribed by means of the SMART technology into cDNA, whereby the original complexity and relative frequency in the amplified DNA is realized. The p68 expression is utilized with a radioactive or, alternatively a fluorescent marked gene-specific DNA probe for hybridization and the resulting profiles selected with a phosphorus imager or a fluorescence scanner. Thereby, the signal strength correlates with the frequency of the p68 specific cDNA molecule and give a clue to the expression strength (mRNA) of the p68 protein helicase in the original tissue probes.

2.4

The protein stability is markedly influenced through the post translational modification determined. p68 polypeptides from tumor tissue and corresponding normal tissue are isolated with anti-p68 antibody and the purified p68 polypeptides undergo a mass spectroscopy analysis. Mass differences can illustrate the exact manner of posttranslational modification, which differentiates the p68 helicase of tumor cells from the normal cells.

These studies are essentially to the determination whether the promoter of the p68 gene is being up regulated (as for example with ER alpha) or whether the p68 polypeptide is stabilized through posttranslational modification (for example through blocking of ubiquitylation through acetylization or sumosylation in the same lysine residues.

Similar experiments are conducted in order to determine the mechanisms on which the p72 and p82 polypeptide over expression in tumors is based.

### 3. p68 Expression in Cell Line Model

3.1

A determination of the RNA helicase polypeptide expression levels illustrates the examination of cell extracts in an immunoblot analysis of proteins separated by electrophoresis. For that, cell lines are utilized that are isolated and established from a hepatocellular, cholangio-, pancreas-, stomach or esophagus carcinoma, such as for example HuCCA-1, Panc 1, ASPC-1, HUP-T3, NC1-N87, AGS KYSE, and corresponding ones, which are isolated not from tumors but from normal tissue, and with SV40 “largeT” transformed cell lines, such as fro example THLE-2 and Het-1A. These cell lines are cultivated under standard cell culture conditions and each viewed as model systems for the described tumor types.

The Expression level of the p68 RNA helicase polypeptide can be determined from the total cell lysate or from lysates of the core, respectively the cytosolic fraction, by means of SDS polyacrylamid gel electrophoresis (SDS-PAGE) and followed by immunoblot with a peroxidase conjugated secondary antibody specifically directed against p68 protein. The expression strength of p68 is then read from the strength of the test reaction (for example ECL system) and correlated with the amount of p68 protein present in the respective cell line. When computed to the number of cells or a reference protein such as for example actine, the p68 expression levels can be compared with each other (cf. FIG. 12 A to H).

A comparison of the signal strength between the carcinogenic and non carcinogenic cell lines indicates the expression level of the p68 RNA-helicase in a tissue probe (cf. 12A to F negative control G and positive control 12H).

3.2

A further possibility fro determining the expression strength of the p68 helicase polypeptides of the cell lines described under 3.1 represents an “immunoassay” (e.g. an ELISA, enzyme linked immunosorbent assay). For that, the total proteins from the cultivated cell lines are isolated (total cell lysate) and immobilized in a ‘multiwell ELISA-plate’. After saturating the protein binding sites with BSA solution, the test of the p68 RNA-helicases is followed with a polyclonal antibody directed against the p68 protein and with a secondary antibody conjugated with an enzyme (e.g. alkaline phosphatase or peroxidase) or fluorescence conjugated. The expression strength of p68 is then read from the strength of the enzymatic test reaction (a substrate conversion leads to a colored product) or the fluorescence intensity in an ‘ELISA reader’ and correlated with the amount of p68 protein present in the examined probe. A comparison of the expression level in the normal tissue shows an overexpression in the carcinogenic tissue.

3.3

In a further embodiment the level of p68 RNA-helicase can be tested by means of a specific antibody (such as a-p68 (555-576), a-p68 (1582) in an immunocytochemical method. For that purpose, the cells of the respective lines are cultivated on glass cover vials and fixed in a subconfluent to confluent condition. After permeabilization of the membrane and saturation of the unspecified protein binding sites in a BSA solution, the p68 protein can be made visible by means of binding specific primary antibodies (a-p68 (555-576), a-p68 (1582), a-p68 1583)) and the fluorescently marked secondary antibodies in a fluorescence microscope. The fluorescence intensity correlates with the expression strength respectively, the frequency of the protein in the cell and can be compared with the signal from other cell lines. Furthermore, the subcellular location (cytosol nucleus) of the p68 protein can also be determined and thus provides information of the normal respectively the abnormal distribution of it in the cell (cf. 13 A to F),

### 4. The Role of the P68 Helicase Polypeptide in Tumor Cells

4.1

The following experiments are conducted in order to investigate the influence of the p68 RNA helicase polypeptide on the tumor cell phenotype. The p68 RNA-helicase polypeptide expression is suppressed in human tumor cell lines through RNA-interference and the thereby resulting changes in the growth rate and the substrate dependent growth determined. In short: the siRNA molecules are potent agents for investigation of the effects of down regulation (“down regulation”) of the RNA helicase polypeptide expression in living cells. In particular, in the case where p68 is a proto oncogene, its down-regulation can inhibit the cell transformation.

For example, established tumor cell lines can be transfected with siRNA molecules with the goal to produce RNA interference for p68 and the resulting effect can be examined through substrate dependent growth (e.g. the “soft agar assay”).

The following human p68 RNA-helicases cDNA sequences can be used as siRNA ‘target’ sequences:

1. TAAGGAAGATTGTGGATCA
2. TAAGACCTGATAGGCAAAC
3. ACCACAACATTGTTCTTCAGAT
4. ACTTATTCGTCTAATTGGAA
5. AGAAGATGTATGAGCTTA
6. GACAGAGGTTCAGGTCGTT
7. GAACTGCTCGCAGTACCA

Furthermore, the substrate dependent growth can be utilized in the “soft agar assay” and the colony-forming assay (“colony formation assay”) in order to examine the capacity of p68 polypeptides and to support the β-catenine mediated transformation of NIH3T3 and RK3E-cells (rat). These investigations can determine whether the p68 RNA-helicase polypeptide is involved in the cell transformation and or the induction thereof.

Similar techniques can be applied in order to reduce the expression of p72 or p82 RNA-helicase. For example, the following human p72 and p82 RNA helicase cDNA sequences can be used as siRNA ‘target’ sequences.

1. GAGACGCTGTGATGATCTG
2. GATGTCAAGTTTGTGATCA

### In Vivo Experiments

Human RKO colon cancer cells were infected with a mixture of two retroviruses. The first retrovirus codes for the expression of a shRNA (small hairpin RNA), which is directed against the human p68, and the second retro virus which encodes the expression of a shRNA, which is directed against a human p72. This shRNA binds also p82. For the purpose of simplifying the nomenclature, following the term “p72/p82” is used when p72 and p82 are meant. Accordingly, “p72/p82” has to be understood a “and/or”.

After three times infecting the RKO cell with this virus mixture (FIG. 11A, right track) or the control viruses (FIG. 11A left track) and following a 48 h growth phase, the protein extracts were produced by using Laemmli Buffer. Illustrated are the respective Western blots of these protein extracts. It is shown that the p68 and p68/p72 proteins are down-regulated more that 80% by the shRNA, as compared to the control protein actine which has not been influenced. Interestingly, cyclin D1 and cMyc are also down-regulated in the presence of p68 and p72/p82 shRNA, which could produce a reduction of cell proliferation. Furthermore, it was observed, that the activated form of the caspase 3 was up-regulated, which can point to an increased apoptosis rate. This points to an overexpression of p68 und/or p72/p82 and can contribute to an increased cell proliferation and protect from apoptosis. Both are features, which would facilitate a tumor development under the co-operation of p68 and p72/p82.

An experiment on cell proliferation also confirmed the already described observations. RKO cell were seeded after infection with a mixture of both retroviruses with a 25% confluence and after 2 days viewed in a phase contrast microscope. While control cell proliferated very strongly and formed an almost confluent cell lawn, the cells infected with p68 and p72/p82 shRNA hardly proliferated. Furthermore many new round cell bodies (which in FIG. 11B are seen as light refracting), and which may point to dying cells. Obviously the proliferation of RKO cells is inhibited by p68 and p72/p82 shRNA.

Also, in an in vivo experiment, the RNA-helicases p68 and p72/p82 were shown to be important for the tumor growth.

(3.5×10⁶) RKO cells were infected with a mixture of both retroviruses (see supra) and followed by injection into the left torso of naked mice. Parallel to this, the experiment was conducted by using control shRNA.

After 32 days, the tumors were isolated and the tumor mass determined. The statistical evaluation of the tumors from each 8 mice in both experiment groups shows distinctly that the tumor growth is strongly inhibited by p68 and p72/p82 shRNA (cf. FIGS. 11C and D).

### REFERENCES

Abdelhaleem, M. (2204). Do Human RNA-helicases have a role in Cancer? Biochimica et Biophysica Acta 1704: 37-46.
Abdelhaleem, M. (2205) RNA-helicases: regulators of differentiation. Clinical Biochemistry 38: 499-503.
Anzick, S L, Kononen, J., Walker, R. L., Azorsa, D. O., Tanner M. M., Guan, X. Y., Sauter, G., Kallioniemi, O. P., Trent, J. M., Meltzer, P. S. (19917) AiB1, a steroid receptor amplified in breast and ovarian cancer. Science 277: 965-968
Causevic, M., Gordon Hislop, R. G., Kernohan, N. M., Carey, F. A., Kay, R. A., Steele, R. J. Fuller-Pace, F. V. (2201) Over expression and poly-ubiquitylation of the DEAD box RNA helicase p68 in colorectal tumours, Oncogene 20: 7734-43.
Goodman, R. H., Smolik, S., (2000) CBP/p300 in cell growth, transformation and development. Genes. Dev. 14:15553-1577.
Gutknecht, R. (2002) The versatile functions of the transcriptional coactivators p 300 and CBP and their roles in disease. Hist. Histopathol. 17: 657-668.
Lanz, R. B., McKenna, N. J., Onate, S. A., Albrecht, U., Wong, J. Tsai, S. Y., M. J. O'Malley, B. W. (1999). As steroid receptor coactivator, SRA functions as an RNA and is present in an SRC-1 complex. Cell 97:17-27.
Rossow, K. L., Janknecht, R. (2003) Synergism between p68 RNA helicase and the transitional coactivators CBP and p300. Oncogene 22:151-156.
Watanabe, M. Yanagisawa, J., Kitagawa, H, Takeyama, K., Ogawa, S., Arao, Y., Suzawa, M., Kobayashi, Y., Yano, T. M. Yoshikawa, H., Masuhiro, Y., Kato. S. (2201) A subfamily of RNA biding DEAD-box proteins acts an estrogen receptor alpha coactivator through the n-terminal activation domain (AF-1) with a RNA coactivator. SRA. EMBO J. 20: 1341-1352.
Yang, L., Lin, C., Liu, Z. R. (2005) Phosphorylations of DEAD-box p68 RNA helicase are associated with cancer development and cell proliferation. Mo. Cancer Res., 3 (6):355-363.

1. Method for the diagnosis of one of the following diseases in a mammal, preferably in a human, characterized in that a probe from the mammal is examined with respect to an elevated level of RNA-helicase, whereby the elevated level in RNA helicase shows the disease: esophagus carcinoma, pancreas carcinoma, stomach carcinoma, hepatocellular carcinoma, hepatoblastoma and cholangiocellular carcinoma.

2. Method according to claim 1, characterized in that the RNA helicase is a DEAD-box helicase.

3. Method according to claim 2, characterized in that the RNA-helicase is a p68 helicase.

4. Method according to claim 2, characterized in that the RNA-helicase is p72 RNA helicase.

5. Method according to claim 2, characterized in that the RNA-helicase is a p82 helicase.

6. Method for treating one of the following diseases in a mammal, preferably in a human, characterized in that that the mammal is administered a an inhibitor for the RNA helicase activity: esophagus carcinoma, pancreas carcinoma, stomach carcinoma, hepatocellular carcinoma, hepatoblastoma and cholangiocellular carcinoma.

7. Method according to claim 6, characterized in that the RNA-helicase is a DEAD-box helicase.

8. Method according to claim 6, characterized in that the RNA-helicase is a p68 helicase.

9. Method according to claim 6, characterized in that the RNA-helicase is p72 RNA helicase.

10. Method according to claim 6, characterized in that the RNA-helicase is a p82 helicase.

11. Method according to claim 6, characterized in that the inhibitor is a siRNA molecule which reduces the expression of the helicase.


SEQUENCE LISTING (13 sequences)

SEQ ID NO: 1 - DNA, 19 bp, Human
taaggaagattgtggatca

SEQ ID NO: 2 - DNA, 19 bp, Human
taagacctgataggcaaac

SEQ ID NO: 3 - DNA, 19 bp, Human
accacaacattcttcagat

SEQ ID NO: 4 - DNA, 19 bp, Human
acttattcgtctaatggaa

SEQ ID NO: 5 - DNA, 19 bp, Human
agaagatgtgatgagctta

SEQ ID NO: 6 - DNA, 19 bp, Human
gacagaggttcaggtcgtt

SEQ ID NO: 7 - DNA, 19 bp, Human
gaactgctcgcagtaccaa

SEQ ID NO: 8 - DNA, 19 bp, Human
gagacgctgtgatgatctg

SEQ ID NO: 9 - DNA, 19 bp, Human
gatgtcaagtttgtgatca

SEQ ID NO: 10 - DNA, 2325 bp, Human
acctcattcatttctaccggtctctagtagtgcagcttcggctggtgtcatcggtgtccttcctccgctgccgcccccgcaaggcttcgccgtcatcgaggccatttccagcgacttgtcgcacgcttttctatatacttcgttccccgccaaccgcaaccattgacgccatgtcgggttattcgagtgaccgagaccgcggccgggaccgagggtttggtgcacctcgatttggaggaagtagggcagggcccttatctggaaagaagtttggaaaccctggggagaaattagttaaaaagaagtggaatcttgatgagctgcctaaatttgagaagaatttttatcaagagcaccctgatttggctaggcgcacagcacaagaggtggaaacatacagaagaagcaaggaaattacagttagaggtcacaactgcccgaagccagttctaaatttttatgaagccaatttccctgcaaatgtcatggatgttattgcaagacagaatttcactgaacccactgctattcaagctcagggatggccagttgctctaagtggattggatatggttggagtggcacagactggatctgggaaaacattgtcttatttgcttcctgccattgtccacatcaatcatcagccattcctagagagaggcgatgggcctatttgtttggtgctggcaccaactcgggaactggcccaacaggtgcagcaagtagctgctgaatattgtagagcatgtcgcttgaagtctacttgtatctacggtggtgctcctaagggaccacaaatacgtgatttggagagaggtgtggaaatctgtattgcaacacctggaagactgattgactttttagagtgtggaaaaaccaatctgagaagaacaacctaccttgtccttgatgaagcagatagaatgcttgatatgggctttgaaccccaaataaggaagattgtggatcaaataagacctgataggcaaactctaatgtggagtgcgacttggccaaaagaagtaagacagcttgctgaagatttcctgaaagactatattcatataaacattggtgcacttgaactgagtgcaaaccacaacattcttcagattgtggatgtgtgtcatgacgtagaaaaggatgaaaaacttattcgtctaatggaagagatcatgagtgagaaggagaataaaaccattgtttttgtggaaaccaaaagaagatgtgatgagcttaccagaaaaatgaggagagatgggtggcctgccatgggtatccatggtgacaagagtcaacaagagcgtgactgggttctaaatgaattcaaacatggaaaagctcctattctgattgctacagatgtggcctccagagggctagatgtggaagatgtgaaatttgtcatcaattatgactaccctaactcctcagaggattatattcatcgaattggaagaactgctcgcagtaccaaaacaggcacagcatacactttctttacacctaataacataaagcaagtgagcgaccttatctctgtgcttcgtgaagctaatcaagcaattaatcccaagttgcttcagttggtcgaagacagaggttcaggtcgttccaggggtagaggaggcatgaaggatgaccgtcgggacagatactctgcgggcaaaaggggtggatttaatacctttagagacagggaaaattatgacagaggttactctagcctgcttaaaagagattttggggcaaaaactcagaatggtgtttacagtgctgcaaattacaccaatgggagctttggaagtaattttgtgtctgctggtatacagaccagttttaggactggtaatccaacagggacttaccagaatggttatgatagcactcagcaatacggaagtaatgttccaaatatgcacaatggtatgaaccaacaggcatatgcatatcctgctactgcagctgcacctatgattggttatccaatgccaacaggatattcccaataagactttagaagtatatgtaaatgtctgtttttcataattgctctttatattgtgtgttatctgacaagatagttatttaagaaacatgggaattgcagaaatgactgcagtgcagcagtaattatggtgcactttttcgctatttaagttggatatttctctacattcctgaaacaatttttaggttttttttgtactagaaaatgcaggcagtgttttcacaaaagtaaatgtacagtgatttgaaatacaataatgaaggcaatgcatggccttccaataaaaaatatttgaagactgaaaaaaaaaa

SEQ ID NO: 11 - protein, 614 aa, Human
MSGYSSDRDRGRDRGFGAPRFGGSRAGPLSGKKFGNPGEKLVKKKWNLDELPKFEKNFYQEHPDLARRTAQEVETYRRSKEITVRGHNCPKPVLNFYEANFPANVMDVIARQNFTEPTAIQAQGWPVALSGLDMVGVAQTGSGKTLSYLLPAIVHINHQPFLERGDGPICLVLAPTRELAQQVQQVAAEYCRACRLKSTCIYGGAPKGPQIRDLERGVEICIATPGRLIDFLECGKTNLRRTTYLVLDEADRMLDMGFEPQIRKIVDQIRPDRQTLMWSATWPKEVRQLAEDFLKDYIHINIGALELSANHNILQIVDVCHDVEKDEKLIRLMEEIMSEKENKTIVFVETKRRCDELTRKMRRDGWPAMGIHGDKSQQERDWVLNEFKHGKAPILIATDVASRGLDVEDVKFVINYDYPNSSEDYIHRIGRTARSTKTGTAYTFFTPNNIKQVSDLISVLREANQAINPKLLQLVEDRGSGRSRGRGGMKDDRRDRYSAGKRGGFNTFRDRENYDRGYSSLLKRDFGAKTQNGVYSAANYTNGSFGSNFVSAGIQTSFRTGNPTGTYQNGYDSTQQYGSNVPNMHNGMNQQAYAYPATAAAPMIGYPMPTGYSQ

SEQ ID NO: 12 - DNA, 2520 bp, Human
gccattttgtgcagtcgctgggaaggaaggagacgcctaaaccgcggcactgcccggtttgagcgtagccaaacctgcccaccggctttgtagccccgattctctgtgttttgctcccgtctccgacgagagaggcggcgacggtggcgtctgcgacgggagacagcgcgtcggagcgagagagcgctgcgcctgccgccgccccaacagcggaggcgccgccgccatcggtcgtcaccagaccggagccgcaggccctcccgagcccggccatccgtgccccgctcccagatctctatccttttgggaccatgcgcggaggaggctttggggaccgggaccgggatcgtgaccgtggaggatttggagcaagaggtggtggtggccttcccccgaagaaatttggtaatcctggggagcgtttgcgtaaaaaaaagtgggatttgagtgagctccccaagtttgagaaaaatttttatgtggaacatccggaagtagcaaggctgacaccatatgaggttgatgagctacgccgaaagaaggagattacagtgagggggggagatgtttgtcctaaacccgtgtttgccttccatcatgctaacttcccacaatatgtaatggatgtgttgatggatcagcactttacagaaccaactccaattcagtgccagggatttccgttggctcttagtggccgggatatggtgggcattgctcagactggctctgggaagacgttggcgtatctcctgcctgcaattgttcatattaaccaccagccatacttggaaaggggagatggcccaatctgtctagttctggctcctaccagagagcttgcccagcaagtacagcaggtggccgatgactatggcaaatgttctagattgaagagtacttgtatttatggaggtgctcctaaaggtccccagattcgagacttggaaagaggtgttgagatctgcatagccactcctggacgtctgatagatttcctggagtcaggaaagacaaatcttcgccgatgtacttaccttgtattggacgaagctgacagaatgcttgatatggggtttgaaccccagatccgtaaaattgttgaccaaatcaggcctgataggcagacactgatgtggagtgcaacctggccaaaagaagtaagacagcttgcagaggatttccttcgtgattacacccagatcaacgtaggcaatctggagttgagtgccaaccacaacatcctccagatagtggatgtctgcatggaaagtgaaaaagaccacaagttgatccaactaatggaagaaataatggctgaaaaggaaaacaaaacaataatatttgtggagacaaagagacgctgtgatgatctgactcgaaggatgcgcagagatggttggccagctatgtgtatccatggagacaagagtcaaccagaaagagattgggtacttaatgagttccgttctggaaaggcacccatccttattgctacagatgtagcctcccgtgggctagatgtggaagatgtcaagtttgtgatcaactatgactatccaaacagctcagaggattatgtgcaccgtattggccgaacagcccgtagcaccaacaagggtaccgcctataccttcttcaccccagggaacctaaaacaggccagagagcttatcaaagtgctggaagaggccaatcaggctatcaatccaaaactgatgcagcttgtggaccacagaggaggcggcggaggcgggggtggtcgttctcgttaccggaccacttcttcagccaacaatcccaatctgatgtatcaggatgagtgtgaccgaaggcttcgaggagtcaaggatggtggccggagagactctgcaagctatcgggatcgtagtgaaaccgatagagctggttatgctaatggcagtggctatggaagtccaaattctgcctttggagcacaagcaggccaatacacctatggtcaaggcacctatggggcagctgcttatggcaccagtagctatacagctcaagaatatggtgctggcacttatggagctagtagcaccacctcaactgggagaagttcacagagctctagccagcagtttagtgggataggccggtctgggcagcagccacagccactgatgtcacaacagtttgcacagcctccgggagctaccaatatgataggttacatggggcagactgcctaccaataccctcctcctcctccccctcctcctccttcacgtaaatgaaaccactcaagtggtagtgactccagcagacttaattacattttaaggaacactgtctttcctttttttttcctcttcgccttttctttttttttccttttttctttttttttttttaatttttccccccaaccatcgtgatttgtcttttcatgcagattagttagaattcactgccaggtttcttctgcccaccaaaatgatccagtctggaataacattttgtaaaaaaaaaaaaaatatatatatatatata

SEQ ID NO: 13 - protein, 729 aa, Human
MPTGFVAPILCVLLPSPTREAATVASATGDSASERESAAPAAAPTAEAPPPSVVTRPEPQALPSPAIRAPLPDLYPFGTMRGGGFGDRDRDRDRGGFGARGGGGLPPKKFGNPGERLRKKKWDLSELPKFEKNFYVEHPEVARLTPYEVDELRRKKEITVRGGDVCPKPVFAFHHANFPQYVMDVLMDQHFTEPTPIQCQGFPLALSGRDMVGIAQTGSGKTLAYLLPAIVHINHQPYLERGDGPICLVLAPTRELAQQVQQVADDYGKCSRLKSTCIYGGAPKGPQIRDLERGVEICIATPGRLIDFLESGKTNLRRCTYLVLDEADRMLDMGFEPQIRKIVDQIRPDRQTLMWSATWPKEVRQLAEDFLRDYTQINVGNLELSANHNILQIVDVCMESEKDHKLIQLMEEIMAEKENKTIIFVETKRRCDDLTRRMRRDGWPAMCIHGDKSQPERDWVLNEFRSGKAPILIATDVASRGLDVEDVKFVINYDYPNSSEDYVHRIGRTARSTNKGTAYTFFTPGNLKQARELIKVLEEANQAINPKLMQLVDHRGGGGGGGGRSRYRTTSSANNPNLMYQDECDRRLRGVKDGGRRDSASYRDRSETDRAGYANGSGYGSPNSAFGAQAGQYTYGQGTYGAAAYGTSSYTAQEYGAGTYGASSTTSTGRSSQSSSQQFSGIGRSGQQPQPLMSQQFAQPPGATNMIGYMGQTAYQYPPPPPPPPPSRK